(19) United States
(12) Patent Application Publication
(10) Pub. No.: US 2025/0325724 A1
(43) Pub. Date: Oct. 23, 2025

(54) A FRAGRANCE DIFFUSER FOR A FLUSH TANK

(71) Applicants: Vijayalakshmi RAGHUNATHAN, Chennai (IN); Valgam Rajagopal RAGHUNATHAN, Chennai (IN)

(72) Inventor: Valgam Rajagopal RAGHUNATHAN, Chennai (IN)

(21) Appl. No.: 18/855,337

(22) PCT Filed: Mar. 24, 2023
(86) PCT No.: PCT/IN2023/050285

§ 371 Date: Oct. 9, 2024

(30) Foreign Application Priority Data
Apr. 16, 2022 (IN) 202241022544

(51) Int. Cl. A61L 9/05

(52) U.S. Cl. CPC A61L 9/05; A61L 2209/131

(57) ABSTRACT

The present invention relates to fragrance diffuser (10) for a flush tank, comprising an enclosure (100) having an operative top surface (102) having an aperture and an operative bottom surface (104). A siphon tube (112) having an outer end (118) and an inner end (120). A filing tube (114) fluidly connected to the siphon tube (112). A canister (108) containing a fragrant composition. The canister (108) comprises a membrane (110) fluidly connected with the exchange chamber (116). A floatable ball (130) is disposed within said enclosure (100), wherein when water fills the flush tank and water level reaches the outer end (118) of the siphon tube (112), water enters the enclosure (100) through the siphon tube (112) and the floatable ball (130) floating on water rises within the enclosure (100) to cover the aperture on the top surface (102) of the enclosure (100).

### FIELD OF THE INVENTION

The present invention relates to the field of diffusers. More particularly, the present disclosure relates to a fragrance diffuser for flush tanks configured to release fragrance whenever the flush tank is operated.

### BACKGROUND OF THE INVENTION

Keeping the toilets clean and fragrant is a known problem in many households. The problem intensifies more where toilets are being used by a significant number of users such as in offices, hotels, educational institutions and other such public places.

It is a challenge to keep the toilets, majorly in public spaces, clean and fragrant all the time as usually after a few hours of continuous usage, the surrounding air gets unpleasant unless of course they are frequently and persistently cleaned, disinfected and where possible, perfumed to counteract unpleasant odors.

Conventionally, handheld spray devices as well as wall mounted push-canister sprays are being used in the toilets to get rid of foul odor. These spray devices contain various fragrances compressed into a canister part of the device, which may be actuated to release a fragrance when required.

Apart from that, there are many devices that are intended to be used as “in the bowl” or “in the cistern” to provide a coloring, cleaning, fragrancing and/or sanitizing effect to toilet bowls. For example, in certain applications, an air freshening device installed within a flush tank produces a cleanser-sanitizer solution with the water stored in a flush tank along with an aerosol spray of deodorizer for a fixed duration in between flushing cycles.

However, the conventional “in-the-tank” odor removing devices for use in toilets have certain drawbacks, some of them are listed as below:
If the toilet is not used for a longer period of time, the amount of disinfectant material inside the device may get overly dissolved with the water inside the flush tank or the fragrance released in a previous cycle may have faded away.
The fragrance element reacting with the disinfectant material may affect the nature of both the compositions.
Both fragrance and the disinfectant material generally release simultaneously, leading to a mixing of their respective fragrances.

Thus there is felt a need for an effective device that not only keeps the toilets hygienic but also fragrant even after multiple/repeated uses without affecting the quality of the disinfectant/fragrant compositions used therein.

### SUMMARY

The present invention relates to a fragrance diffuser for flush tanks attached toilets.

In accordance with one aspect of the invention, the diffuser comprises an enclosure having an operative top surface and an operative bottom surface and a side wall therebetween defining the enclosure. The operative top surface includes an aperture which allows the entry of air from the top of the diffuser. The fragrance diffuser includes a siphon tube disposed within said enclosure, the siphon tube having an outer end and an inner end, wherein the outer end is configured for inflow of water from the flush tank into the enclosure through the inner end, and the inner end is configured for outflow of water from the enclosure to the flush tank through the passage. The enclosure further includes a filing tube disposed within said enclosure, along one of the side walls of the enclosure with a bottom passage fluidly connected to the siphon tube and top passage fluidly connected to an exchange chamber. The enclosure further includes a canister containing a liquid fragrant composition therein which is water insoluble and lesser density than water and disposed inside said enclosure. The canister comprises a water soluble membrane on a portion of said canister, the membrane fluidly connected with the exchange chamber. The enclosure further includes a floatable ball disposed within the enclosure. When water fills the flush tank and water level reaches the outer end of the siphon tube, water enters into the enclosure through the siphon tube and the floatable ball floating on water rises within the enclosure to cover the aperture on the top surface of the enclosure.

In an embodiment, the exchange chamber has a vent at a top section of the exchange chamber.

In an embodiment, the fragrance diffuser is fixed on a side wall of the flush tank.

In an embodiment, the siphon tube traverses to a space adapted to store a disinfectant at a base of said diffuser.

In an embodiment, the filling tube comprises an atomizer positioned at the bottom passage of the filling tube.

In an embodiment, the membrane of the canister is a water soluble membrane disposed between the canister and the exchange chamber.

### BRIEF DESCRIPTION OF INVENTION

For a fuller understanding of the invention, reference should be made to the following detailed description, taken in connection with the accompanying drawings, in which:

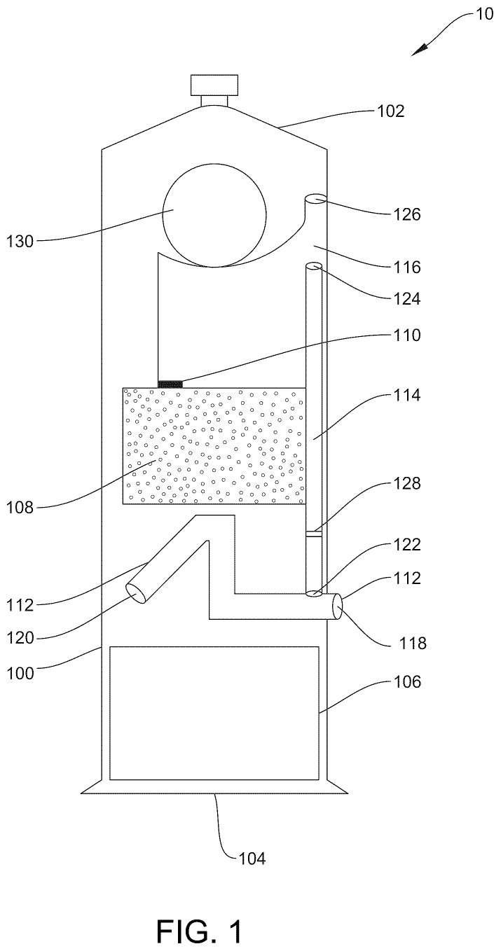

FIG. 1 illustrates a cross-sectional view of a fragrance diffuser for a flush tank, as an embodiment of the present invention.

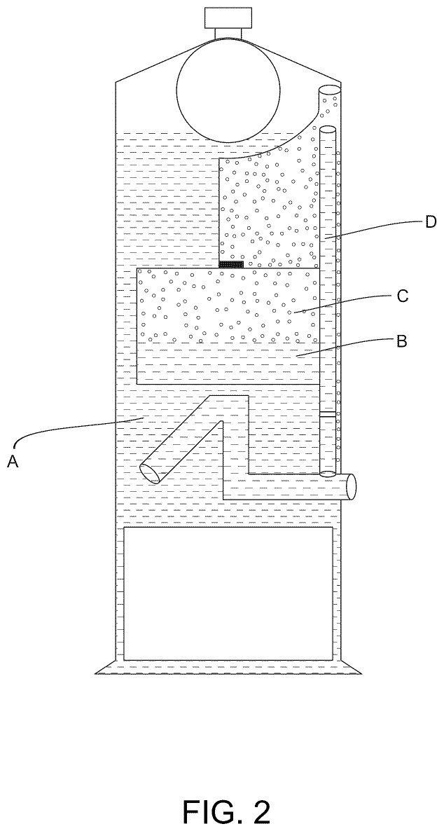

FIG. 2 illustrates a sectional view of the fragrance diffuser in a water-filled state, as an embodiment of the present invention.

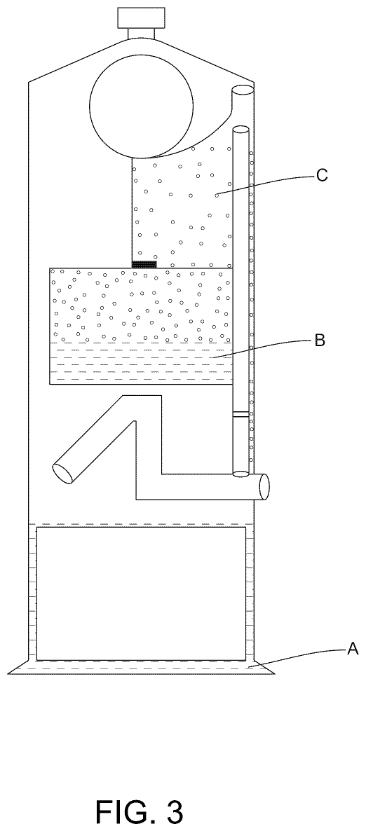

FIG. 3 illustrates a sectional view of a fragrance diffuser after flushing, as an embodiment of the present invention.

### LIST OF REFERENCE NUMERALS

| Elements | Reference numeral |
|---|---|
| Fragrance diffuser | 10 |
| Enclosure | 100 |
| Operative top surface | 102 |
| Operative bottom surface | 104 |
| Disinfectant material | 106 |
| Canister | 108 |
| Water-soluble sealing membrane | 110 |
| Siphon tube | 112 |
| Outer end of siphon tube | 118 |
| Inner end of siphon tube | 120 |
| Filling tube | 114 |
| Exchange chamber | 116 |
| Bottom end of filling tube | 122 |
| Top end of filling tube | 124 |
| Vent of the exchange chamber | 126 |
| Atomizer | 128 |
| Floatable ball | 130 |

All referenced methods are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicants in no way disclaim these technical aspects, and it is contemplated that the claimed invention may encompass one or more of the technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

In the following detailed description of the preferred embodiments, reference is made to the accompanying drawings, which form a part thereof, and within which are shown by way of illustration specific embodiments by which the invention may be practiced. It is to be understood that other embodiments may be utilized, and structural changes may be made without departing from the scope of the invention.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

The present disclosure herein below discloses a fragrance diffuser that aims to ameliorate one or more disadvantages associated with conventional “in-the-tank” type fragrance diffusers. FIG. 1 illustrates a cross-sectional view of a fragrance diffuser 10 for a flush tank, in accordance with an embodiment of the present invention. The fragrance diffuser 10 is configured to be positioned within the flush tank, preferably attached to an internal side wall of the flush tank. In an embodiment, the fragrance diffuser device 10 includes an enclosure 100 having an operative top surface 102, an operative bottom surface 104 and a sidewall encompassing the bottom and top surfaces, thereby defining the enclosure 100. In an embodiment, the operative top surface 102 has a through-hole/aperture that lets displacement of air out of the canister when the diffuser gets submerged in water inside the flush tank.

In an embodiment, a disinfectant material 106 is provided near a bottom of the diffuser 10. In an embodiment, the disinfectant material 106 is a solid composition placed at the bottom of the enclosure 100.

Further, in an embodiment, the enclosure 100 encloses a canister 108 positioned at a height from the bottom surface 104, preferably attached to an inside of the side wall of the enclosure 100. The canister 108 comprises a fragrant material. In an embodiment, the canister 108 includes at least one surface, a portion of which is water permeable so as to allow ingress of water inside the canister 108. In an embodiment, a water-soluble sealing membrane 110 secures the fragrant material inside the canister 108 till the time the fragrance diffuser 10 is not in contact with water. The water-soluble sealing membrane 110 secures the fragrant fluid during the transport and handling, and prior to installation.

Further, in an embodiment, the enclosure 100 includes a siphon tube 112 disposed within the enclosure 100 in a manner so as to allow seepage of flush tank water inside the enclosure 100. Preferably, the siphon tube 112 is an open ended tube having an outer end 118 opening outside the enclosure 100 and an inner end 120 opening within the enclosure 100. In an embodiment, the siphon tube 112 positioned just above the space containing the disinfectant material 106. The enclosure 100 further includes a filling tube 114 disposed within the enclosure 100 in fluid communication with the siphon tube 112. The filling tube 114, in an embodiment, is positioned along a sidewall of the enclosure and meets the siphon tube 112 near the outer end 118.

In particular, the filling tube 114 lets the water rise vertically upwards inside the tube to a height equal to a level of the water within the enclosure 100. In an embodiment, a bottom end 122 of the filling tube 114 is fluidly attached to the siphon tube 112 while a top end 124 of the filling tube 114 is open to let the rising water inside the filling tube 114 to fall therefrom onto the canister 108. The filling tube 114 includes an atomizer 128, positioned near a bottom of the filling tube 114. In an embodiment, the atomizer 128 lets the water exit the filling tube 114 in an atomized form when a flushing action takes place. This enables a controlled mixing of the fragrant water coming from the filling tube 114 with the disinfectant water coming out from the siphon tube 112.

In an embodiment, a space above the canister 108 is partially enclosed to form an exchange chamber 116, which is configured such that the sealing member 110 and the filling tube 114 lie within the space forming the exchange chamber 116. Preferably, the exchange chamber 116 is attached to the side wall of the enclosure 100 on a side where the canister 108 and the filling tube 114 are positioned. Top end of the exchange chamber 116 has an open vent 126, positioned just above the open top end 124 of the filling tube 114.

Further, the fragrance diffuser 10 includes a floatable ball 130. The floatable ball 130 may lie either on top of the canister 108 or on the curve surface of the exchange chamber 116 in idle state. In an embodiment, a top of the exchange chamber 116 is designed to let the floatable ball 130 rest thereon sealing the hole.

FIGS. 2 and 3 illustrate working embodiments of the diffuser, in water-filled state and after flushing state, respectively shown in FIG. 1, in accordance with different embodiments of the present invention. When the flush tank gets filled with water, the same gets seep into the enclosure 100 through the siphon tube 112 as well. In an embodiment, the water enters into the diffuser 10 through the outer end 118 of siphon tube 112 and starts filling up the space within the enclosure 100. Simultaneously, the water rises in the filling tube 114 as well. The water entering the enclosure 100 through the siphon tube and starts submerging the disinfectant material 106 first and then rises further to make the floating ball rise as well. The floatable ball 130 rises to a point where it comes into contact with the through hole at the operative top surface 102, encircling it fully to seal and prevent any further passage of air therefrom. At this stage, when floatable ball 130 blocks the top hole of the enclosure 100, the water seepage inside the enclosure gets stopped and a level of water is set just around a height equalling, a height of the filling tube 114. At this stage, some amount of water rising inside the filling tube 114 flows into the exchange chamber 116 and fall on a top surface of the canister 108.

In an embodiment, the water entering the exchange chamber 116 comes in contact with the water-soluble sealing membrane, thus dissolving it and eventually entering inside of the canister 108. The water entering into the canister 108 comes in contact with a fragrant material stored within the canister 108, the water eventually starts displacing the fragrant material equal to an amount of water entering the canister 108. The fragrant material, being lighter in density, starts rising upwards and spreads around an area just above a level of liquid inside the exchange chamber 116. When a user operates the flush, the water within the enclosure also starts moving out of enclosure through the siphon tube with the emptying of the flush tank, taking along both the disinfectant material as well as the fragrant material through the siphon tube separately. In this manner, the fragrant material is released in a controlled manner along with the disinfectant material.

FIGS. 2 and 3 show the fragrance diffuser 10 before and after flushing of the flush tank, respectively.

The reference numerals A, B, C and D refer to water (shown as dashed lines) and/or fragrant aerosol (shown as bubbles) inside the enclosure 100 and other sub-components positioned therein.

The enclosure 100 as disclosed above keeps the fragrant material as well as the disinfectant material separate during packaging and/or transportation of the fragrance diffuser. By keeping them separate, any chemical reaction between the two materials is also avoided. Moreover, the device lets a pre-controlled amount of fragrance mix with the flushing water, thus avoiding wastage and making efficient use of the diffuser.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A fragrance diffuser (10) for a flush tank, the fragrance diffuser (10) disposed within the flush tank, the fragrance diffuser (10) comprising:
an enclosure (100) having an operative top surface (102) and an operative bottom surface (104) and a side wall therebetween defining said enclosure (100), the top surface (102) having an aperture, the fragrance diffuser (10) comprising:
a siphon tube (112) disposed within said enclosure (100), the siphon tube (112) having an outer end (118) and an inner end (120), wherein the outer end (118) is configured for inflow of water from the flush tank into the enclosure (100) through the inner end (120), and the inner end (120) is configured for outflow of water from the enclosure (100) to the flush tank through the outer end (118);
a floatable ball (130) disposed within said enclosure (100), wherein when water fills the flush tank and water level reaches the outer end (118) of the siphon tube (112), water enters the enclosure (100) through the siphon tube (112) and the floatable ball (130) floating on water rises within the enclosure (100) to cover the aperture on the top surface of the enclosure (100);
a filling tube (114) disposed within said enclosure (100), along one of the side walls of the enclosure (100) with a bottom passage (122) fluidly connected to the siphon tube (112) and top passage (124) connected to an exchange chamber (116); and
a canister (108) containing a fragrant composition therein, and having a density less than that of water, disposed within said enclosure (100), wherein said canister (108) comprises a water soluble membrane (110) in a portion of said canister (108), the membrane (110) fluidly connected with the exchange chamber (116), wherein the water entering the canister displaces a corresponding volume of the fragrant composition.

2. The fragrance diffuser (10) as claimed in claim 1, wherein the exchange chamber (116) has a vent (126) at a top section of the exchange chamber (116).

3. The fragrance diffuser (10) as claimed in claim 1, wherein the fragrance diffuser (10) is fixed on a side wall of the flush tank.

4. The fragrance diffuser (10) as claimed in claim 1, wherein the siphon tube (112) traverses to a space adapted to store a disinfectant (106) at a base of said diffuser (10).

5. The fragrance diffuser (10) as claimed in claim 1, wherein the filling tube (114) comprises an atomizer (128) positioned at the bottom passage (122) of the filling tube (114).

6. The fragrance diffuser (10) as claimed in claim 1, wherein the membrane (110) of the canister (108) is a water soluble membrane (110) disposed between the canister (108) and the exchange chamber (116).
